(19) United States
(12) Patent Application Publication
(10) Pub. No.: US 2004/0093006 A1
(43) Pub. Date: May 13, 2004

(54) Balloon catheter

(76) Inventor: Alain Lecuyer, Grasse (FR)

Correspondence Address: Lawrence M. Nawrocki, NAWROCKI, ROONEY & SIVERTSON, P.A., Broadway Place East, Suite 401, 3433 Broadway Street Northeast, Minneapolis, MN, 55413, US

(21) Appl. No.: 10/402,352

(22) Filed: Mar. 27, 2003

(30) Foreign Application Priority Data
Mar. 27, 2002 (FR) 02/03850

(51) Int. Cl. A61M 29/00

(52) U.S. Cl. 606/194

(57) ABSTRACT

A catheter having a distal end and a proximal end, a balloon defining an opening sealingly connected to the distal end of the catheter, a connector connecting the proximal end of the catheter to an injector for injecting a balloon-inflation fluid with the catheter, an electrode fixed to the balloon, generally opposite to its opening for connection with the catheter, and an electrically conductive wire having a distal end and a proximal end, passing through the catheter and electrically connected at its distal end to the electrode and at its proximal end to an electrical generator, the wire passing through the balloon and having its distal end fixed to the electrode, a lock being provided for locking the sliding of the wire inside the catheter.

The present invention concerns a balloon catheter and more particularly a balloon catheter of the type comprising a catheter having a distal end and a proximal end, a balloon comprising an opening sealingly connected to the distal end of the catheter, means of connecting the proximal end of the catheter to means of injecting a balloon-inflation fluid by means of the catheter, an electrode fixed to the balloon, generally opposite to its opening for connection with the catheter, and an electrically conductive wire having a distal end and a proximal end, passing through the catheter and electrically connected at its distal end to the electrode and at its proximal end to means of connection to an electrical generator.

In neurosurgery operations are known during which it is necessary to perforate a membrane. It may be a case for example of perforating the floor of the third cerebral ventricle in the treatment of hydrocephalus.

These operations were traditionally performed in two stages. Firstly, the membrane was opened, for example using an electrocoagulator, which was then withdrawn. A balloon catheter was then introduced in its place, which was inflated in order to dilate the opening.

Such a procedure resulted firstly in a loss of time, always prejudicial during an operation. In addition, a discharge of cerebrospinal fluid could also occur when the electrocoagulator was withdrawn.

This is the reason why, in the document EP-A-868 883, a balloon catheter was proposed for neurosurgery comprising a catheter at the end of which a dilation balloon was mounted, the catheter being arranged so as to be connected to means of inflating the balloon and also comprising inside it an electrical supply wire for an electrode forming an electrocoagulation tip fixed beyond the balloon.

More particularly, the end of the body coaxially supported a metallic tube at the end of which the electrocoagulation tip was welded and to which the supply wire was electrically connected, the balloon being mounted around the metallic tube, whose wall was pierced to enable the balloon to be inflated.

Thus the catheter, or tubular body, described in this prior document fulfils two functions. Firstly it serves as a conduit for the balloon inflation fluid. Secondly it forms a sheath for the supply wire to the electrocoagulation tip.

The membrane is then perforated in a single operation. The catheter being put in place with the electrocoagulation tip in contact with the membrane to be perforated, this tip is powered and therefore produces an opening in the membrane. The catheter is then slightly pressed in so as to partially engage the balloon in the opening. Finally, this balloon is inflated so as to dilate the opening, after which the catheter is withdrawn.

The known arrangement generally gives satisfaction for relatively large diameters, for example greater than 1 mm. It is on the other hand difficult to obtain smaller diameters, having regard to the presence of the metallic tube supporting the electrocoagulation tip.

The present invention aims to mitigate this drawback.

More particularly, the aim of the invention is to provide a balloon catheter of the type stated above but whose diameter can be much less.

To this end, the object of the invention is a balloon catheter comprising a catheter having a distal end and a proximal end, a balloon comprising an opening sealingly connected to the distal end of the catheter, means of connecting the proximal end of the catheter to means of injecting a balloon-inflation fluid by means of the catheter, an electrode fixed to the balloon, generally opposite to its opening for connection with the catheter, and an electrically conductive wire having a distal end and a proximal end, passing through the catheter and electrically connected at its distal end to the electrode and at its proximal end to means of connection to an electrical generator, the said wire passing through the said balloon and being fixed to the said electrode, means being provided for locking the sliding of the said wire inside the catheter.

In a particular embodiment, this balloon catheter comprises a portion of tube over which the distal end of the catheter and the opening in the balloon are engaged and fixed in a sealed manner, the said portion of tube not substantially extending inside the balloon, and the said wire passing through the said tube portion whilst being fixed to the tube.

Thus the tube serves only to provide the sealed connection between the distal end of the catheter and the balloon rather than supporting the coagulation electrode. It is therefore not added to the diameter of the balloon. The electrode is directly supplied electrically by the wire, which also gives rigidity to the end of the balloon catheter in order to facilitate its insertion.

In addition, the wire being fixed to the tube, it cannot slide in the tube and catheter when pressure is exerted on the electrode with a view to its insertion.

Finally, it is no longer necessary to make tricky piercings in the wall of the tube, as was the case in the prior art.

In a particular embodiment, the said tube is made from deformable material, and the wire is fixed to it by crimping.

The electrode can be crimped to the distal end of the wire.

The distal end of the catheter can be adhesively bonded to the external surface of the tube portion.

Likewise, the opening of the balloon can be adhesively bonded to the external surface of the tube portion.

In addition, the distal end of the catheter and the opening in the balloon can be adhesively bonded to each other.

In a particular embodiment of the invention, the balloon is generally tubular and has a second opening opposite to its opening for connection to the distal end of the catheter, said second opening being adhesively bonded sealingly to the said electrode.

Also in a particular embodiment, the balloon has a restriction in diameter between its opening for connection to the distal end of the catheter and the point of connection of the electrode, so as to prevent it from sliding on one side or other of the opening when it is inflated.

A particular embodiment of the invention will now be described by way of non-limiting example with reference to the accompanying schematic drawings, in which:

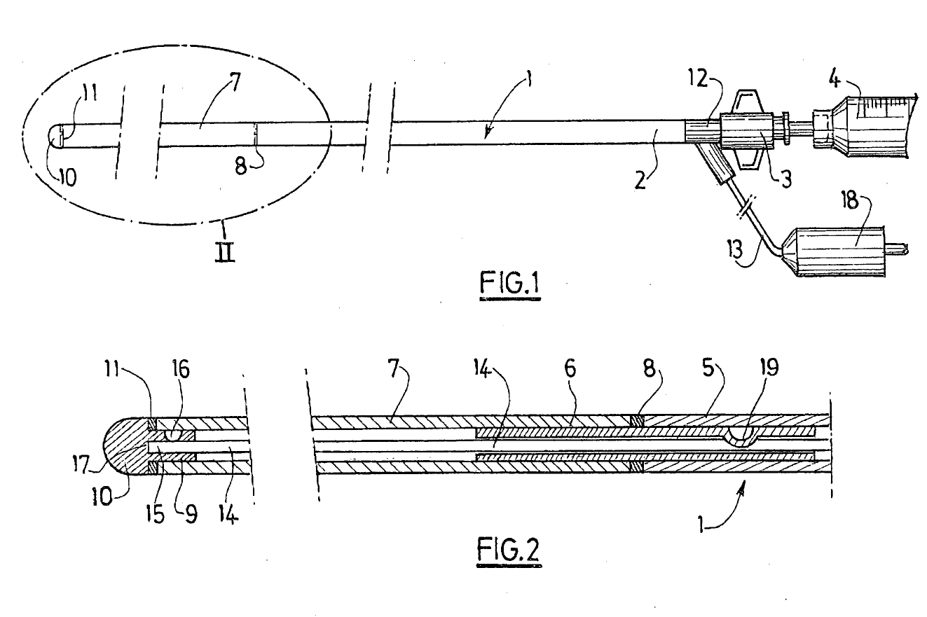

FIG. 1 is a side view of a balloon catheter according to the invention in which the diameter of the catheter proper has been increased for more clarity; and

FIG. 2 is an enlarged view in section of the detail 11 in FIG. 1.

This balloon catheter comprises a tubular catheter 1 provided at its proximal end 2 with an inflation connector 3. The connector 3 is here adapted to receive the connecting piece of a syringe 4.

At its other end 5, the catheter 1 is sealingly connected to a tube portion 6, for example made from stainless steel, by adhesive bonding or any other suitable means, the catheter and the tube portion being coaxial and one end of the tube portion being engaged in the distal end 5 of the catheter 1.

A tubular silicone balloon 7 has one of its openings engaged on the other end of the tube portion 6. This balloon 7 is adhesively bonded to the tube portion 6 or sealingly fixed by any other means. The tube portion 6 does not substantially enter the balloon 7.

A ring of adhesive 8 provides the connection between the distal opening of the catheter 1 and the opening in the balloon which is facing.

The balloon 7 also comprises in its middle part a restriction in diameter whose function will be disclosed below.

The other opening of the balloon 7 is engaged on a cylindrical projection 9 of an electrode 10. The balloon 7 is adhesively bonded or fixed by any other suitable means to the projection 9 and a joint of adhesive 11 is disposed between the opening in the balloon and the shoulder on the electrode. Opposite to the projection 9, the electrode 10 is substantially hemispherical.

The catheter 1 forms, close to the connector 3, a coupling 12 entered sealingly by an electrically conductive cable 13. The conductive wire 14, relatively rigid, of this cable 13 is extended throughout the catheter 1, the tube portion 6 and the balloon 7, and its distal end 15 is finally crimped at 16 in a hole 17 formed in the projection 9.

The external end of the cable 13 is connected to a standard connector 18 for a single-pole electrocoagulant probe. Thus the electrode 10 is electrically connected to the connector 18.

The wire 14 is also crimped at 19 in the tube portion 6 so as to prevent its sliding in the catheter when the latter is inserted. The crimping is however such that it enables the inflation fluid to circulate between the wire 14 and the tube portion 6.

To perforate a membrane, the electrode 10 is firstly brought in contact with it, and is supplied from the connector 18. An opening thus being produced in the membrane, the end of the catheter is engaged therein until the membrane is situated level with the restriction in diameter of the balloon 7.

The balloon 7 is then inflated by means of the syringe 4 using the connector 3 in order to dilate the opening in the membrane. Because of the restriction in diameter, the balloon cannot slide on one side or other of the membrane and therefore remains in position.

When the opening has reached a sufficient size, the balloon 7 is deflated and the catheter can be withdrawn.

1. A balloon catheter comprising a catheter (1) having a distal end (5) and a proximal end (2), a balloon (7) comprising an opening sealingly connected to the distal end of the catheter (1), means (3) of connecting the proximal end of the catheter (1) to means (4) of injecting a balloon-inflation fluid (7) by means of the catheter (1), an electrode (10) fixed to the balloon (7), generally opposite to its opening for connection with the catheter (1), and an electrically conductive wire (14) having a distal end (15) and a proximal end, passing through the catheter (1) and electrically connected at its distal end to the electrode (10) and at its proximal end to means (18) of connection to an electrical generator, characterised by the fact that the said wire passes through the said balloon and has its distal end fixed to the said electrode (10), means (19) being provided for locking the sliding of the said wire inside the catheter.

2. A balloon catheter according to claim 1, comprising a portion of tube (6) over which the distal end of the catheter (1) and the opening in the balloon (7) are engaged and fixed in a sealed manner, the said portion of tube (6) not substantially extending inside the balloon (7), and the said wire (14) passing through the said tube portion (6) whilst being fixed to the tube.

3. A balloon catheter according to claim 2, in which the said tube portion (6) is made from deformable material and the wire (14) is fixed to it by crimping.

4. A balloon catheter according to any one of claims 1 to 3, in which the electrode (10) is crimped to the distal end (15) of the wire (14).

5. A balloon catheter according to any one of claims 2 to 4, in which the distal end (5) of the catheter (1) is adhesively bonded to the external surface of the tube portion (6).

6. A balloon catheter according to any one of claims 2 to 5, in which the opening in the balloon (7) is adhesively bonded to the external surface of the tube portion (6).

7. A balloon catheter according to any one of claims 1 to 6, in which the distal end (5) of the catheter (1) and the opening in the balloon (7) are adhesively bonded to each other.

8. A balloon catheter according to any one of claims 1 to 7, in which the balloon (7) is roughly tubular and has a second opening opposite to its opening for connection to the distal end (5) of the catheter (1), the said second opening being adhesively bonded in a sealed fashion to the said electrode (10).

9. A balloon catheter according to any one of claims 1 to 8, in which the balloon (7) has a restriction in diameter between its opening for connection to the distal end (5) of the catheter (1) and the point (16) where the electrode (10) is connected.
